(19) United States
(12) Patent Application Publication
(10) Pub. No.: US 2012/0239131 A1
(43) Pub. Date: Sep. 20, 2012

(54) METHODS AND APPARATUS FOR TREATMENT OF ANEURYSMAL TISSUE

(75) Inventors: Oluwashola S. Sulaimon, Santa Rosa, CA (US); Ayala Hezi-Yamit, Santa Rosa, CA (US); Susan Rea-Peterson, Santa Rosa, CA (US); Mingfei Chen, Santa Rosa, CA (US)

(73) Assignee: Medtronic Vascular, Inc., Santa Rosa, CA (US)

(21) Appl. No.: 13/421,617

(22) Filed: Mar. 15, 2012

Related U.S. Application Data
(60) Provisional application No. 61/452,952, filed on Mar. 15, 2011.

(51) Int. Cl. A61F 2/06; A61F 2/82

(52) U.S. Cl. 623/1.13; 623/1.42

(57) ABSTRACT

Methods and apparatus for aiding aneurysm repair are provided. Such apparatus is constructed to support or bolster the aneurysmal site and supply a therapeutic agent to aid in healing the surrounding aneurysmal tissue.

### CROSS-REFERENCE TO RELATED APPLICATIONS

This application claims the benefit and priority of U.S. Provisional Application No. 61/452,952 filed Mar. 15, 2011, entitled “Method and Apparatus for Treatment of Aneurysmal Tissue” and is herein incorporated by reference for all purposes.

### BACKGROUND

Aneurysms, such as abdominal aortic aneurysm (AAA), are a complex vascular disease with multifactorial processes leading to aneurysm formation, growth and rupture. An aneurysm typically occurs when weakened areas of a vascular wall (e.g., abdominal aortic wall) results in ballooning of the blood vessel of at least 1.5 times its normal diameter, or greater than 3 centimeters (cm) diameter in total. The cause of death is typically a ruptured aneurysm following progressive weakening and dilation of the aneurysmal sac.

Current medical management options for large AAA, for example, are either open aortic repair (OAR), endovascular aneurysm repair (EVAR), or follow-up by imaging at intervals (i.e., conservative treatment with no therapeutic intervention). OARs involves laparatomy and insertion of a prosthetic graft to replace the aneurysmal aorta. EVAR, which has revolutionized the treatment of AAA, involves a minimally invasive approach by placement of an endoluminal stent graft (ELG) through a transfemoral approach. Despite the increasing numbers of EVAR procedures over OAR, a major limitation with EVAR is that this treatment modality only provides a mechanical resolution and does not address the molecular and cellular processes/pathways involved in the underlying disease pathophysiology.

### SUMMARY

The present disclosure provides methods and devices for treating a vascular aneurysm, such as an abdominal aortic aneurysm. This involves, for example, addressing the problem of chronic inflammation and continued breakdown of aortic aneurysm tissue. Such methods and devices support or bolster the aneurysmal site and supply a combination of therapeutic agents to aid in healing the surrounding aneurysmal tissue.

Embodiments according to the present disclosure provide localized application of therapeutic agents useful to reduce the severity and the progression of an aneurysm at an aneurysmal site. Certain embodiments include the administration of two or more therapeutic agents as described herein using local delivery. The agents preferably are localized to (adjacent or within) the aneurysmal site by the placement of an intravascular treatment device that is comprised of, or within which is provided, the therapeutic agents.

In certain embodiments, the present disclosure provides a method of treating a vascular aneurysm (e.g., abdominal, thoracic, and cerebral aneurysm, particularly an abdominal aortic aneurysm) in a subject, the method comprising: providing an intravascular treatment device comprising two or more therapeutic agents, wherein the two or more therapeutic agents comprise: at least one HMG-CoA reductase inhibitor; and at least one (preferably at least two, and more preferably at least three) of a therapeutic agent selected from the group consisting of an ACE inhibitor, an Angiotensin II Receptor Blocker, a calcium channel blocker, a renin inhibitor, a prostanoid receptor antagonist, a cholesterol absorption inhibitor, and combinations thereof; and positioning the intravascular treatment device in the interior of an aneurysmal site in a blood vessel, wherein the intravascular treatment device supports the aneurysmal site upon deployment.

In certain embodiments, the present disclosure provides an intravascular treatment device locatable interior of an aneurysmal site in a blood vessel; wherein the device supports the aneurysmal site upon deployment, contracts when the aneurysmal site contracts, and comprises two or more therapeutic agents, wherein the two or more therapeutic agents comprise: at least one HMG-CoA reductase inhibitor; and at least one (preferably at least two, and more preferably at least three) of a therapeutic agent selected from the group consisting of an ACE inhibitor, an Angiotensin II Receptor Blocker, a calcium channel blocker, a renin inhibitor, a prostanoid receptor antagonist, a cholesterol absorption inhibitor, and combinations thereof.

In certain embodiments, the HMG-CoA reductase inhibitor is a statin.

In certain embodiments, the ACE inhibitor is selected from the group consisting of trandolapril, lisinopril, enalapril, ramipril, fosinopril, cilazapril, imidapril, captopril, quinapril, perindopril, benazepril, moexipril, physiologically active metabolites thereof, and combinations thereof.

In certain embodiments, the Angiotensin II Receptor Blocker is selected from the group consisting of irbestartan, candesartan, losartan, valsartan, telmisartan, eprosartan, olmesartan, physiologically active metabolites thereof, and combinations thereof.

In certain embodiments, the calcium channel blocker is selected from the group consisting of amlodipine, aranidipine, azelnidipine, barnidipine, benidipine, cilnidipine, clevidipine, isradipine, efonidipine, felodipine, lacidipine, lercanidipine, manidipine, nicardipine, nifedipine, nilvadipine, nimodipine, nisoldipine, nitrendipine, pranidipine, physiologically active metabolites thereof, and combinations thereof.

In certain embodiments, the renin inhibitor is selected from the group consisting of aliskiren, remikiren, enalkiren, MK8141, physiologically active metabolites thereof, and combinations thereof.

In certain embodiments, the prostanoid receptor antagonist is laropiprant, an azaindole, physiologically active metabolites thereof, and combinations thereof.

In certain embodiments, the a cholesterol absorption inhibitor is selected from the group consisting of ezetimibe, niacin, and Niemann-Pick Cl-Like 1 (NPC1L1) inhibitors, physiologically active metabolites thereof, and combinations thereof.

The term “treating” in the context of “treating an abdominal aortic aneurysm” means improving the condition of, or reducing the severity of, a vascular aneurism (e.g., an aortic aneurysm). This includes aiding aneurysm repair by addressing, for example, the problem of continued breakdown of aortic aneurysm tissue and the progression of the aneurysm. Thus, inhibition of further development of an aneurysm is included within the term “treating.” This term also encompasses altering the pathophysiology and encouraging tissue incorporation into a graft or stent graft, for example, for sealing of the graft or stent graft to the tissue to prevent leakage of blood into the aneurysmal site.

The term “comprises” and variations thereof do not have a limiting meaning where these terms appear in the description and claims.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

As used herein, “a,” “an,” “the,” “at least one,” and “one or more” are used interchangeably. Thus, for example, a device that comprises “a” polymer can be interpreted to mean that the device includes “one or more” polymers.

As used herein, the term “or” is generally employed in its usual sense including “and/or” unless the content clearly dictates otherwise.

The term “and/or” means one or all of the listed elements or a combination of any two or more of the listed elements (e.g., preventing and/or treating an affliction means preventing, treating, or both treating and preventing further afflictions).

Also herein, all numbers are assumed to be modified by the term “about” and preferably by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used.

Also herein, the recitations of numerical ranges by endpoints include all numbers subsumed within that range (e.g., 1 to 5 includes 1, 1.5, 2, 2.75, 3, 3.80, 4, 5, etc.) including the endpoints.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

FIG. 1 is a sectional view of a descending aorta with a stent graft placed therein.

FIG. 2 illustrates one example of an endoluminal stent graft including therapeutic agents described herein located within a proximal anchor region and a distal anchor region.

FIG. 3 is a chart of aortic diameter measurements by ultrasonography. Telmisartan and irbesartan were effective in inhibiting aneurysm development. Fluvastatin showed good inhibition of AAA when compared to control and doxycycline. Doxycycline historically used as an inhibitor of AAA; did not inhibit AAA growth in this model.

FIG. 4 is a graph showing the relationship between aneurysm incidence and passage of time. Aneurysm was defined as either the presence of dissection or more than 50% increase in diameter. Telmisartan and irbesartan inhibited aneurysm development well.

FIG. 5 is a bar chart of plasma drug content evaluated by high performance liquid chromatography (HPLC).

### DETAILED DESCRIPTION

The present disclosure provides methods and devices for treating a vascular aneurysm such as an abdominal, thoracic, and cerebral aneurysm, particularly an abdominal aortic aneurysm (AAA). Such methods and devices support or bolster the aneurysmal site and supply a combination of therapeutic agents to treat (e.g., to aid in healing) the surrounding aneurysmal tissue.

Applicants have discovered that the pathogenesis of AAA suggests the following mechanisms play a concurrent role in the formation of aneurysms: 1) aortic wall proteolysis by matrix metalloproteinases (MMPs); 2) chronic aortic wall inflammation; 3) revascularization in the arterial media (angiogenesis); 4) smooth muscle cell (SMC) apoptosis; and 5) oxidative stress. Pharmacologically targeting one or more of these mechanisms offers a convenient alternative to surgical intervention alone. Treatments that inhibit or alter AAA pathophysiology may ultimately change the management of AAA disease in humans and supplement endovascular intervention.

Thus, the present disclosure is directed to the use of therapeutic agents that target one or more of these mechanisms. Preferably, two or more therapeutic agents are used in combination in a treatment protocol. More preferably, three or more therapeutic agents are used in combination in a treatment protocol. These may be used in admixture, e.g., in a mixture of therapeutic agents in a polymer coating on an intravascular treatment device. Alternatively, they may be used in combination, but not in an admixture. For example, they may be applied to different portions of an intravascular treatment device.

The therapeutic agents for use in the present disclosure include an HMG-CoA reductase inhibitor, an ACE inhibitor, an Angiotensin II Receptor Blocker (ARB), a calcium channel blocker, a renin inhibitor, a prostanoid receptor antagonist, and a cholesterol absorption inhibitor (i.e., cholesterol lowering agent other than a statin). They may be in the form or a salt, a free base, a solvate, a prodrug, or a physiologically active metabolite. They may be in the form of physiologically active compounds and compositions containing such compounds; and their prodrugs, and pharmaceutically acceptable salts and solvates of such compounds and their prodrugs, as well as novel compounds within the scope of formula of these compounds

Preferably, at least one therapeutic agent is an HMG-CoA reductase inhibitor.

In certain embodiments, the present disclosure provides a method of treating an aneurysm (preferably an abdominal aortic aneurysm) in a subject, the method comprising: providing an intravascular treatment device comprising two or more therapeutic agents, wherein the two or more therapeutic agents comprise: at least one HMG-CoA reductase inhibitor; and at least one of a therapeutic agent selected from the group consisting of an ACE inhibitor, an ARB, a calcium channel blocker, a renin inhibitor, a prostanoid receptor antagonist, and a cholesterol absorption inhibitor, and combinations thereof; and positioning the intravascular treatment device in the interior of an aneurysmal site in a blood vessel, wherein the intravascular treatment device supports the aneurysmal site upon deployment. Thus, compounds from at least two different classes of therapeutic agents are used. In certain preferred embodiments, compound from at least three different classes of therapeutic agents are used.

Embodiments according to the present disclosure provide localized application of therapeutic agents useful to reduce the severity and the progression of an aneurysm at an aneurysmal site. Certain embodiments include the administration of two or more therapeutic agents as described herein using local delivery. The agents are localized to (e.g., adjacent or within) the aneurysmal site (e.g., within the aneurysmal sac or at a neck region of the aneurysm) by the placement of an intravascular treatment device that is comprised of, or within which is provided, the therapeutic agents.

The therapeutic agents (typically, two or more, and preferably, three or more) can be incorporated directly into an intravascular treatment device (e.g., incorporated into a polymer for forming a graft, placed inside such a double-walled stent graft) or into a carrier associated with an intravascular treatment device (e.g., as a coating or in a pouch), or both. Typically, the therapeutic agents are delivered by the intravascular treatment device over time to the local tissue. The materials to be used for such a carrier can be synthetic organic polymers, natural organic polymers, inorganics, or combinations of these. The physical form of the therapeutic agent/carrier formulation can be a film, sheet, coating, slab, gel, capsule, microparticle, nanoparticle, or combinations of these.

In certain embodiments, the carrier is placed in a pouch that is attached to or wrapped around the outer—i.e., blood vessel wall side—of a stent graft passing through an aneurysmal blood vessel. The stent graft isolates the aneurysmal region of the blood vessel from blood flow and provides a structure on which to attach the delivery device so that the agent may be delivered directly to the aneurysmal blood vessel site. The delivery device is positioned on or wrapped around a stent, graft, stent graft, or other intervention device (all referred to herein as intravascular treatment devices) spanning the aneurysmal site through the interior of a blood vessel to release therapeutic agents into the space between the intervention device and the wall of the aneurysmal blood vessel.

### Therapeutic Agents

Using an ApoE^{-/-}+ANG II mouse model of AAA, Applicants have identified disease relevant molecular targets which were modulated by therapeutic intervention. Mechanistic data show that potential molecular and cellular targets for AAA treatment fall within the following categories (i) inhibition of inflammatory processed (ii) inhibition of protease and ECM (Extra cellular matrix) degradation pathways, (iii) suppression of oxidative stress, and (iv) augmenting ECM formation pathways.

The classes of compounds which have been selected are based on the similarity in their mode of action (MOA) in inhibiting molecular pathways implicated in the pathophysiology of AAA as identified in our drug screening study and also the fact that most AAA patient have existing cardiovascular co-morbidities such as atherosclerosis, hypertension (HTN), congestive heart failure (CHF), myocardial infarct (MI) to mention but a few. Furthermore, given the atherosclerosis is a risk factor for AAA, reduction in cholesterol from statins may exhibit beneficial effects on AAA due to the pleiotrophic effects of statins include an anti-inflammatory effect, anti-oxidative effect, and the reduction of MMP secretion.

HMG-CoA ([beta]-hydroxy[beta]-methylglutaryl coenzyme A) reductase inhibitors are a class of drug used to lower cholesterol levels by inhibiting the enzyme HMG-CoA reductase, which plays a central role in the production of cholesterol in the liver. Increased cholesterol levels have been associated with cardiovascular diseases (CVD), so HMG-CoA reductase inhibitors, particularly statins, are used in the prevention of these diseases. Randomized controlled trials have shown that they are most effective in those already suffering from cardiovascular disease (secondary prevention), but they are also advocated and used extensively in those without previous CVD but with elevated cholesterol levels and other risk factors (such as diabetes and high blood pressure) that increase a person's risk.

Exemplary statins include lovastatin, cerivastatin, pitavastatin, pravastatin, fluvastatin, rosuvastatin, simivastatin, and atorvastatin. The best-selling of the statins is atorvastatin, marketed as Lipitor and manufactured by Pfizer. By 2003 it had become the best-selling pharmaceutical in history. As of 2010, a number of other statins came on the market: fluvastatin (Lescol); lovastatin (Mevacor, Altocor, Altoprev); pitavastatin (Livalo, Pitava); pravastatin (Pravachol, Selektine, Lipostat); rosuvastatin (Crestor); and simvastatin (Zocor, Lipex). Various combinations of these compounds can be used if desired.

Angiotensin II is a very potent chemical that causes muscles surrounding blood vessels to contract, thereby narrowing blood vessels. This narrowing increases the pressure within the vessels and can cause high blood pressure (hypertension). Angiotensin II receptor blockers (ARBs) are medications that block the action of angiotensin II by preventing angiotensin II from binding to angiotensin II receptors on blood vessels. As a result, blood vessels enlarge (dilate) and blood pressure is reduced. Reduced blood pressure makes it easier for the heart to pump blood and can improve symptoms of heart failure. In addition, the progression of kidney disease due to high blood pressure or diabetes is slowed. ARBs have effects that are similar to angiotensin converting enzyme (ACE) inhibitors, but ACE inhibitors act by preventing the formation of angiotensin II rather than by blocking the binding of angiotensin II to muscles on blood vessels.

ACE inhibitors are known to alter vascular wall remodeling, and are used widely in the treatment of hypertension, congestive heart failure, and other cardiovascular disorders. In addition to ACE inhibitors' antihypertensive effects, these compounds are recognized as having influence on connective tissue remodeling after myocardial infarction or vascular wall injury. ACE inhibitors prevent the generation of Angiotensin II, and many of the effects of Angiotensin II involve activation of cellular ATI receptors.

The essential effect of ACE inhibitors is to inhibit the conversion of relatively inactive angiotensin Ito the active angiotensin II. Thus, ACE inhibitors attenuate or abolish responses to angiotensin I but not to angiotensin II. In this regard, ACE inhibitors are highly selective drugs. They do not interact directly with other components of the angiotensin system, and the principal pharmacological and clinical effects of ACE inhibitors seem to arise from suppression of synthesis of angiotensin II.

ACE is a rather nonspecific enzyme and cleaves dipeptide units from substrates with diverse amino acid sequences. Preferred substrates have only one free carboxyl group in the carboxyl-terminal amino acid, and proline must not be the penultimate amino acid. Although slow conversion of angiotensin I to angiontensin II occurs in plasma, the very rapid metabolism that occurs in vivo is due largely to the activity of membrane-bound ACE present on the luminal aspect of the vascular system—thus, the localized delivery of the ACE inhibitor contemplated by the present disclosure provides a distinct advantage over systemic modes of administration.

Many ACE inhibitors have been synthesized: however, a majority of ACE inhibitors are ester-containing prodrugs that are 100 to 1000 times less potent ACE inhibitors than the active metabolites but have an increased bioavailability for oral administration than the active molecules. In general, ACE inhibitors differ with regard to three properties: (1) potency; (2) whether ACE inhibition is due primarily to the drug itself or to conversion of a prodrug to an active metabolite; and (3) pharmacokinetics (i.e., the extent of absorption, effect of food on absorption, plasma half-life, tissue distribution, and mechanisms of elimination). For example, with the notable exceptions of fosinopril and spirapril, which display balanced elimination by the liver and kidneys, ACE inhibitors are cleared predominantly by the kidneys. Therefore, impaired renal function inhibits significantly the plasma clearance of most ACE inhibitors, and dosages of such ACE inhibitors should be reduced in patients with renal impairment.

Examplary ACE inhibitors include trandolapril (including its active metabolite trandolaprilat), lisinopril, enalapril (including its active metabolite enalaprilat), ramipril (including its active metabolite ramiprilat), fosinopril (including its active metabolite fosinoprilat), cilazapril, imidapril, captopril, quinapril (including its active metabolite quinaprilat), perindopril (including its active metabolite perindoprilat), benazepril (including its active metabolite benazeprilat), and moexipril (and its active metabolite moexiprilat). Combinations of these can be used if desired.

For systemic administration there is no compelling reason to favor one ACE inhibitor over another, since all ACE inhibitors effectively block the conversion of angiotensin I to angiontensin II and all have similar therapeutic indications, adverse-effect profiles and contraindications. However, there are preferred ACE inhibitors for use in the present disclosure. ACE inhibitors differ markedly in their activity and whether they are administered as a prodrug, and this difference leads to preferred locally delivered ACE inhibitors according to the present disclosure.

One preferred ACE inhibitor is captopril (Capoten). Captopril was the first ACE inhibitor to be marketed, and is a potent ACE inhibitor with a Ki of 1.7 nM. Captopril contains a sulfhydryl moiety. Given orally, captopril is rapidly absorbed and has a bioavailability of about 75%.

Another preferred ACE inhibitor is lisinopril. Lisinopril (Prinivil, Zestril) is a lysine analog of enalaprilat (the active form of enalapril). Unlike enalapril, lisinopril itself is active. In vitro, lisinopril is a slightly more potent ACE inhibitor than is enalaprilat, and is slowly, variably, and incompletely (about 30%) absorbed after oral administration; peak concentrations in the plasma are achieved in about 7 hours. Lisinopril is cleared as the intact compound in the kidney, and its half-life in the plasma is about 12 hours. Lisinopril does not accumulate in the tissues.

Enalapril (Vasotec) was the second ACE inhibitor approved in the United States. However, because enalapril is a prodrug that is not highly active and must be hydrolyzed by esterases in the liver to produce enalaprilat, the active form, enalapril is not a preferred ACE inhibitor of the present disclosure. Similarly, fosinopril (Monopril), benazepril (Lotensin), fosinopril (Monopril), trandolapril (Mavik), quinapril (Accupril), ramipril (Altace), moexipirl (Univasc) and perindopril (Aceon) are all prodrugs that require cleavage by hepatic esterases to transform them into active, ACE-inhibiting forms, and are not preferred ACE inhibitors. However, the active forms of these compounds (I.e., the compounds that result from the prodrugs being converted by hepatic esterases)—namely, enalaprilat (Vasotec injection), fosinoprilat, benazeprilat, trandolaprilat, quinaprilat, ramiprilat, moexiprilat, and perindoprilat—are suitable for use, and because of the localized drug delivery, the bioavailability issues that affect the oral administration of the active forms of these agents are moot.

ARBs are used for controlling high blood pressure, treating heart failure, and preventing kidney failure in people with diabetes or high blood pressure. They may also prevent diabetes and reduce the risk of stroke in patients with high blood pressure and an enlarged heart. ARBs may also prevent the recurrence of atrial fibrillation. Since these medications have effects that are similar to those of ACE inhibitors, they often are used when ACE inhibitors are not tolerated by patients (for example, due to excessive coughing).

Exemplary ARBs suitable for use in the present disclosure include irbestartan (Avapro), candesartan (Atacand), losartan (Cozaar), valsartan (Diovan), telmisartan (Micardis), eprosartan (Tevetan), and olmesartan (Benicar). Various combinations of these could be used if desired.

Calcium channel blockers (GCBs) can also be used in methods and devices of the present disclosure. They are a class of drugs and natural substances that disrupt the movement of calcium (Ca²⁺) through calcium channels. CCBs have effects on many excitable cells of the body, such as cardiac muscle, smooth muscles of blood vessels, or neurons. The most widespread clinical usage of calcium channel blockers is to decrease blood pressure in patients with hypertension, with particular efficacy in treating elderly patients. Also, calcium channel blockers frequently are used to control heart rate, prevent cerebral vasospasm, and reduce chest pain due to angina pectoris.

Calcium channel blockers work by blocking voltage-gated calcium channels (VGCCs) in cardiac muscle and blood vessels. This decreases intracellular calcium leading to a reduction in muscle contraction. In the heart, a decrease in calcium available for each beat results in a decrease in cardiac contractility. In blood vessels, a decrease in calcium results in less contraction of the vascular smooth muscle and therefore an increase in arterial diameter (GCBs do not work on venous smooth muscle), a phenomenon called vasodilation. Vasodilation decreases total peripheral resistance, while a decrease in cardiac contractility decreases cardiac output. Since blood pressure is determined by cardiac output and peripheral resistance, blood pressure drops. Calcium channel blockers are especially effective against large vessel stiffness, one of the common causes of elevated systolic blood pressure in elderly patients.

With a relatively low blood pressure, the afterload on the heart decreases; this decreases how hard the heart must work to eject blood into the aorta, and so the amount of oxygen required by the heart decreases accordingly. This can help ameliorate symptoms of ischemic heart disease such as angina pectoris. Unlike β-blockers, calcium channel blockers do not decrease the responsiveness of the heart to input from the sympathetic nervous system. Since moment-to-moment blood pressure regulation is carried out by the sympathetic nervous system (via the baroreceptor reflex), calcium channel blockers allow blood pressure to be maintained more effectively than do n-blockers.

There are several classes of calcium channel blockers, including dihydropyridine calcium channel blockers and non-dihydropyridine calcium channel blockers. Dihydropyridine calcium channel blockers are often used to reduce systemic vascular resistance and arterial pressure, but are not used to treat angina (with the exception of amlodipine, nicardipine, and nifedipine, which carry an indication to treat chronic stable angina as well as vasospastic angina) because the vasodilation and hypotension can lead to reflex tachycardia. This CCB class is easily identified by the suffix “-dipine” and includes amlodipine (Norvasc), aranidipine (Sapresta), azelnidipine (Calblock), barnidipine (HypoCa), benidipine (Coniel), cilnidipine (Atelec, Cinalong, Siscard), clevidipine (Cleviprex), isradipine (DynaCirc, Prescal), efonidipine (Landel), felodipine (Plendil), lacidipine (Motens, Lacipil), lercanidipine (Zanidip), manidipine (Calslot, Madipine), nicardipine (Cardene, Carden SR), nifedipine (Procardia, Adalat), nilvadipine (Nivadil), nimodipine (Nimotop), nisoldipine (Baymycard, Sular, Syscor), nitrendipine (Cardif, Nitrepin, Baylotensin), and pranidipine (Acalas). Various combinations of these can be used if desired.

One class of non-dihydropyridine calcium channel blockers includes phenylalkylamine calcium channel blockers. These are relatively selective for myocardium, reduce myocardial oxygen demand and reverse coronary vasospasm, and are often used to treat angina. They have minimal vasodilatory effects compared with dihydropyridines and therefore cause less reflex tachycardia, making it appealing for treatment of angina, where tachycardia can be the most significant contributor to the heart's need for oxygen. Therefore, as vasodilation is minimal with the phenylalkylamines, the major mechanism of action is causing negative inotropy. Examples include verapamil (Calan, Isoptin) and gallopamil. Combinations of these can be used if desired.

Another class of non-dihyropyridine calcium channel blockers includes benzothiazepine calcium channel blockers. These are an intermediate class between phenylalkylamine and dihydropyridines in their selectivity for vascular calcium channels. By having cardiac depressant and vasodilator actions, benzothiazepines are able to reduce arterial pressure without producing the same degree of reflex cardiac stimulation caused by dihydropyridines. An example of a benzothiazepine is diltiazem (Cardizem).

While most of the calcium channel blockers listed above are relatively selective, there are additional agents that are considered nonselective, including for example, mibefradil, bepridil, fluspirilene, and fendiline. Combinations of these can be used if desired.

Various combinations of any calcium channel blockers could be used if desired.

Renin inhibitors can also be used in methods and devices of the present disclosure. They are compounds used primarily in the treatment of hypertension. They act on the juxtaglomerular cells of kidney, which produce renin in response to decreased blood flow. Renin is an enzyme that plays a major role in the Renin-Angiotensin System, a regulatory system in the body, which is responsible for maintaining homeostasis of blood pressure. The enzyme belongs to the family of aspartic proteases and is responsible for the conversion of inactive angiotensinogen to angiotensin I (Ang I). Angiotensin I by itself is inactive; however, when acted upon by angiotensin converting enzyme (ACE) it gets converted to angiotensin II, which is active and is responsible for most of the pressor effects. Conversion of angiotensinogen to angiotensin I is the rate determining step of the system. The catalytic role played by renin is implicated in mediating blood pressure by the Renin-Angiotensin System.

Direct renin inhibition offers a pharmacological tool in the treatment of hypertension. One example of a direct renin inhibitor is Aliskiren, which is used as an antihypertensive drug. Aliskiren, is an oral renin inhibitor. It is an octanamide, is the first known representative of a new class of completely non-peptide, low-molecular weight, orally active transition-state renin inhibitors. It is a specific in vitro inhibitor of human renin (IC50 in the low nanomolar range), with a plasma half-life of approximately 24 hours. Aliskiren has good water solubility and low lipophilicity and is resistant to biodegradation by peptidases in the intestine, blood circulation, and the liver. Its trade name is Tekturna in the USA, and Rasilez in the UK. Other renin inhibitors are completely different in structure, having a piperidine ring. Ketopiperazine-based renin inhibitors are known. More recently a new series of renin inhibitors based on the ketopiperazine structure was developed. These molecules have a 3,9-diazabicyclo[3.3.1]nonene group in place of the ketopiperazine group.

Examples of renin inhibitors include aliskiren, remikiren, enalkiren, and MK8141. Various combinations of these could be used if desired.

Prostanoid receptor (DP, EP1, EP2) antagonists can also be used in methods and devices of the present disclosure. They are structurally related to the natural agonist or are “non-prostanoid” (often acyl-sulphonamides) compounds. A series of indole-based antagonists of the PGD₂ receptor subtype 1 (DP1 receptor) have been identified. One example is Laropiprant (pINN; codenamed MK-0524A), which is an investigational treatment for hypercholesterolemia, marketed by Merck & Co. as a combination with niacin (tradenames Cordaptive and Tredaptive). Other examples include azaindoles, their physiologically active forms and compositions containing such compounds. Various combinations of prostanoid receptor antagonists can be used if desired.

Cholesterol absorption inhibitors can also be used in methods and devices of the present disclosure. Two organs primarily control cholesterol levels in blood: the liver, which produces cholesterol and bile acids (used to digest fats), and the intestine, which absorbs cholesterol both from food and from the bile. While statins primarily lower cholesterol by preventing its production in the liver, a class of drug called cholesterol absorption inhibitors lowers cholesterol by preventing it from being absorbed in the intestine. These include, ezetimibe, niacin, and Niemann-Pick Cl-Like 1 (NPC1L1) Inhibitors. Various combinations of such compounds can be used if desired.

Ezetimibe acts by decreasing cholesterol absorption in the intestine. It is used alone (marketed as Zetia or Ezetrol), when other cholesterol-lowering medications are not tolerated, or together with statins (e.g., ezetimibe/simvastatin, marketed as Vytorin and Inegy) when statins alone do not control cholesterol. Ezetimibe localizes at the brush border of the small intestine, where it inhibits the absorption of cholesterol from the intestine. Specifically, it appears to bind to a critical mediator of cholesterol absorption, the Niemann-Pick C1-Like 1 (NPC1L1) protein on the gastrointestinal tract epithelial cells as well as in hepatocytes.

Niacin (also known as vitamin B₃, nicotinic acid and vitamin PP) is an organic compound with the formula C₆H₅NO₂. This colorless, water-soluble solid is a derivative of pyridine, with a carboxyl group (COON) at the 3-position. Other forms of vitamin B₃ include the corresponding amide, nicotinamide (“niacinamide”), where the carboxyl group has been replaced by a carboxamide group (CONH₂), as well as more complex amides and a variety of esters. The terms niacin, nicotinamide, and vitamin B₃ are often used interchangeably to refer to any member of this family of compounds, since they have the same biochemical activity. In pharmacological doses, niacin has been proven to reverse atherosclerosis by reducing total cholesterol, triglycerides, very-low-density lipoprotein (VLDL), and low-density lipoprotein (LDL), and increasing high-density lipoprotein (HDL). It has been proposed that niacin has the ability to lower lipoprotein(a), which is beneficial at reducing thrombotic tendency. Niacin also increases the level of high-density lipoprotein (HDL) or “good” cholesterol in blood, and therefore it is sometimes prescribed for patients with low HDL, who are also at high risk of a heart attack.

The dosage of the therapeutic agents described herein will vary depending on the manner in which they are locally delivered. For example, this can depend on the properties of the coating or structure they are incorporated into, including its time-release properties, whether the coating is itself biodegradable, and other properties. Also, the dosage of the therapeutic agents used will vary depending on the potency, pathways of metabolism, extent of absorption, half-life, and mechanisms of elimination of the therapeutic agent itself. In any event, the practitioner is guided by skill and knowledge in the field, and embodiments according to the present disclosure include without limitation dosages that are effective to achieve the described phenomena.

### Intravascular Treatment Devices

Intravascular treatment devices useful in the present disclosure for local delivery of therapeutic agents for the treatment of aneurysms as described herein include endoluminal stent grafts or other intervention devices including vascular stents, coronary artery stents, peripheral vascular stents, cerebral aneurysm filler coils, vascular patches, grafts, and the like.

Various stent grafts and other intravascular treatment devices can be modified using the therapeutic agents described herein using the teachings of the present disclosure. Examples of such intravascular treatment devices include those described, for example, in U.S. Pat. Nos. 6,306,141; 6,911,039; 7,105,016; 7,264,632; 7,655,034; 5,190,546; 6,306,141; 6,911,039; 7,105,016; and 5,871,536; as well as U.S. Patent Publication Nos. 2005/0043786; 2006/0004441; 2007/0032852; and 2007/0239267.

Various methods of incorporating the therapeutic agents into an intravascular treatment device can be used. For example, the therapeutic agents can be incorporated directly into an intravascular treatment device (e.g., incorporated into a polymer for forming a graft) or into a carrier associated with such intravascular treatment device (e.g., as a coating or in a pouch), or both. Typically, the therapeutic agents are delivered by the intravascular treatment device over time to the local tissue. The materials to be used for such a carrier can be synthetic organic polymers, natural organic polymers, inorganics, or combinations of these. The physical form of the therapeutic agent/carrier formulation can be a film, sheet, coating, slab, gel, capsule, microparticle, nanoparticle, or combinations of these.

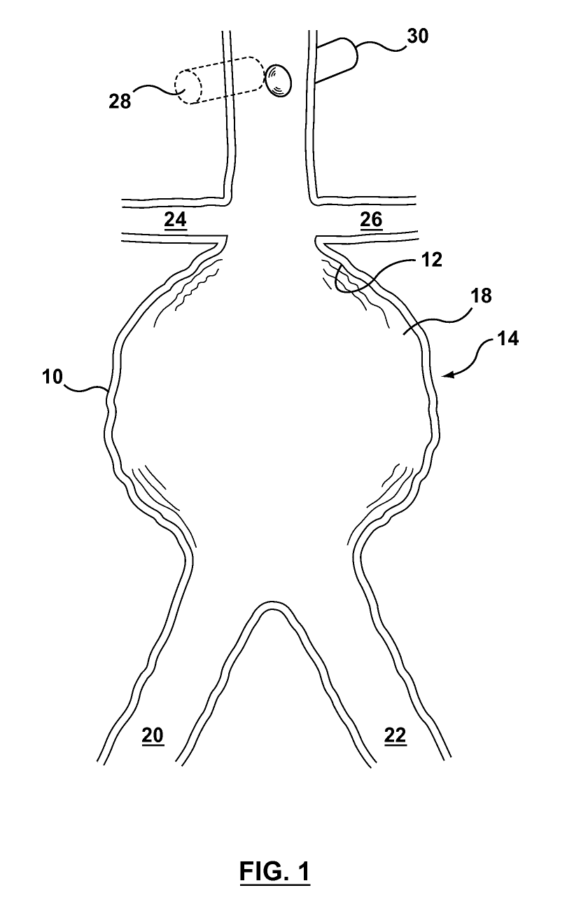

Referring to FIG. 1, there is shown, in section, an aneurysmal blood vessel, in this instance a descending aorta 10. Aorta 10 includes a wall 12, having a healthy wall portion 14 and an aneurysmal wall portion, wherein the aneurysmal wall portion 16 occurs where the aorta has a diameter substantially larger than it does where the healthy wall portion 14 occurs. Aneurysmal portion forms an aneurysmal bulge or sac 18, wherein the elastin in the extra-cellular matrix of the aortic vessel wall 12 is degraded, preventing the aortic wall 12 at the aneurysmal portion from holding the aorta at its healthy diameter against the pressure of blood.

Where the aneurysmal sac 18 has progressed to a diameter on the order of more than twice to three times the diameter of the healthy aortic wall 14, intervention to prevent rupture of the aneurysm is dictated. Surgical intervention can include highly invasive procedures, where the section of the aorta undergoing the aneurysmic event is opened up or removed completely, and a synthetic graft is sewn in place between healthy sections of the aorta or the severed ends of the aorta (not shown). Alternatively, intervention may encompass exclusion of the aneurysmal sac 18 by placement of an exclusion device such as a stent graft 22 (a modular bifurcated stent graft being shown here). The stent graft typically includes a stent portion 24, having a supportive yet collapsible construction (here in a grid pattern), to which a graft portion 26 is sewn or attached. The stent portion 24 provides a tubular body having a support capability sufficient to hold the graft portion 26 in an open position across the aneurysmal sac 18, such that the opposed ends are received and sealed against healthy portions 14 of the of the aorta. The graft portion 26 blocks the passage of blood to the aneurysmal sac 18, and provides a conduit for blood flow past the aneurysmal sac 18.

Preferred endoluminal stent grafts typically include a graft material supported by a stent structure. Generally, endoluminal stent grafts are formed in a tubular shape with proximal and distal neck openings to allow for blood flow. Conventionally, the proximal end of the endoluminal stent graft is referenced with respect to the end closest to the heart (via the length of blood traveled from the heart). Some endoluminal stent grafts further include openings or bifurcations to accommodate lateral branches off the main vessel.

In many embodiments, two or more therapeutic agents described herein, are provided in a delivery vehicle included with an excluding device or intravascular repair vehicle, for example, a stent graft. Referring to FIG. 1, the placement of the stent graft 22 in the aorta 10 is a technique well known to those skilled in the art, and essentially includes the opening of a blood vessel in the leg, and the insertion of the stent graft 22 contained in a catheter into the vessel, guiding the catheter through the vessel, and deploying the stent graft 22 in a position spanning the aneurysmal sac 18.

Implantation of endoluminal stent grafts can be subject to a number of technical problems with subsequent morbidity and mortality. In some patients, the aneurysm neck is diseased and is not a smooth surface; the proximal neck of certain prior art endoluminal stent grafts do not heal and affix properly to these non-smooth luminal walls. This failure of the endoluminal stent graft to incorporate itself at the aneurysm neck (i.e., lack of healing) could allow an endoluminal stent graft to dislodge and migrate distally causing blood flow and pressure leakage into the aneurysm sac, thereby increasing the likelihood of rupture associated with such a Type I leak. In patients having aneurysms with severe neck angularity and/or those with an aortic neck shorter than 10 mm, incomplete contact surface with the vessel wall can produce insufficient anchoring forces for the endoluminal stent graft.

In certain embodiments of the present disclosure an endoluminal stent graft includes two or more of the therapeutic agents discussed above located within at least a proximal anchor region, a distal anchor region, or both. Preferably, the two or more therapeutic agents are located within a proximal anchor region of the endoluminal stent graft. When correctly positioned within a vessel, the therapeutic agents promote cellular growth and allow the vessel wall to heal to the endoluminal sent graft.

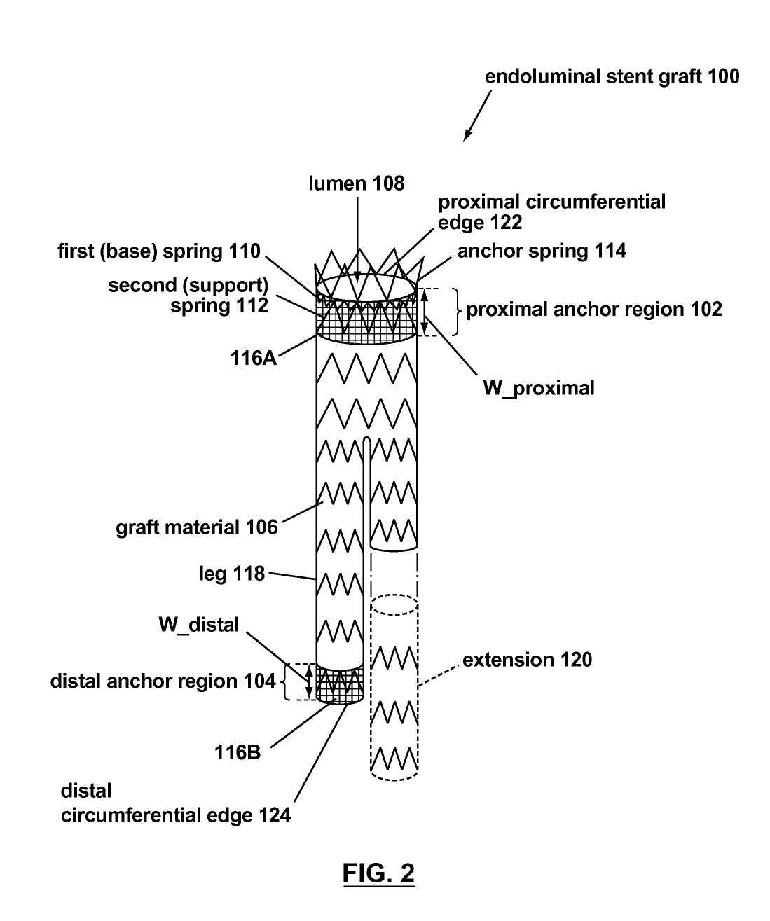

FIG. 2 illustrates one exemplary embodiment of an intravascular treatment device of the present disclosure that can be used for local delivery of the therapeutic agents described herein. This illustrates one example of an endoluminal stent graft 100 including therapeutic agents 116A, 1168 located at selected positions on or in a graft material 106. In certain embodiment the therapeutic agents can be located between two layers of the graft material. Alternatively, the therapeutic agents can be coated on the fibers, or otherwise incorporated into the fibers, of the graft material. Various other methods as would be known to one of skill in the art could be used to incorporate therapeutic agents in or on the graft material at these locations.

Endoluminal stent graft 100, herein termed simply stent graft 100, includes: a graft material 106, i.e., a first material; therapeutic agents at locations 116A, 1168 positioned about an exterior circumferential surface of the first material, and a stent structure of shaped springs, such as a first (base) spring 110, a second (support) spring 112, and an anchor spring 114, among others, distributed within stent graft 100 and attached to graft material 106. Stent graft 100 is shaped to form a lumen 108 that bifurcates distally to accommodate lateral vessels, e.g., the common iliac arteries. Optionally, an extension 120 is included as part of stent graft 100 for some applications.

The stent can be made using nitinol or stainless steel, for example, in the form of a helical configuration with one to three helixes, with drug coatings on the stent; or the stent can be made of biodegradable or non-biodegradable polymers (as described herein below). Thus, in certain embodiments the intravascular treatment device comprises a structural polymeric component comprising the two or more therapeutic agents.

Typically, graft material 106 is a material formed to limit the leakage of blood through graft material 106. Examples of graft material 106 include substantially non-porous fabrics, such as low profile system (LPS) material, or densely knitted fabrics. In certain embodiments, the graft material is a plain weave, 10-40 denier multifilament, woven material. In certain embodiments, the graft material is a twill weave, 10-40 denier monofilament, woven material formed into a flat sheet. In certain embodiments, the graft material is a plain weave, 20-40 denier multifilament, woven material formed into a flat sheet and calendered. A wide variety of the commonly used graft materials are suitable for use herein for any of the embodiments.

As illustrated, proximal anchor region 102 is located at a proximal neck of stent graft 100, and therapeutic agents at location 116A form a right circular cylinder around stent graft 100 within proximal anchor region 102 on an exterior circumferential surface of graft material 106. In this example, proximal anchor region 102 extends longitudinally from a proximal circumferential edge 122 longitudinally toward the distal end of stent graft 100 a specified distance W_proximal along an outer circumferential surface of stent graft 100. W_proximal should be in contact with tissue (endothelium inner layer of the vessel). Therefore, W_proximal should be, ideally, a distance equals to the aneurysm neck (AAA). This distance is usually determined in the individual patient by echography (ultrasonography) or Computed Tomography imaging (CT scanning, CT Scan). In one example, specified distance W_proximal defines a length of what is commonly referred to as the proximal neck of stent graft 100.

Distal anchor region 104 is located at a distal neck of leg 118 of stent graft 100, and therapeutic agents at location 1168 is attached to leg 118 within a distal anchor region 104 on an exterior circumferential surface of graft material 106 of leg 118. In this example, distal anchor region 104 extends from a distal circumferential edge 124 of leg 118 a specified longitudinal distance W_distal towards the proximal end of stent graft 100 and along an outer circumferential surface of leg 118. In the distal part of the graft the presumption is that the graft is substantially in contact with the inner endothelium tissue of the iliac artery. If this is indeed the case, then W_distance is chosen to be in the range of 5-10 mm. In one example, specified distance W_distal defines a length of what is commonly referred to as the distal neck of leg 118 of stent graft 100.

Thus, a group of stent grafts can be provided having a range of specified distances W_proximal and/or distances W_distal so that the range of specified distances corresponds to the range of aneurysm necks commonly encountered in patients. A physician chooses a particular stent graft in the group based on the characteristics of the aneurysm neck in a particular patient.

Particularly preferred embodiments of the present disclosure include two or more of the therapeutic agents described herein (preferably from two or more different classes of therapeutic agents, more preferably from three or more different classes of therapeutic agents) located at the proximal neck of a stent graft.

In other embodiments, the present disclosure provides a delivery device or vehicle to deliver locally therapeutic agents at the site of an aneurysm, e.g., a pouch adjacent to an aneurysmal sac. Referring again to FIG. 1, although the stent graft 22 provides an exclusionary environment through which blood may flow past the aneurysmal sac 18, certain embodiments of the present disclosure involve treating the aneurysmal sac 18. In particular, it is known that fresh blood may leak into the aneurysmal sac 18 region, despite the presence of stent graft 22, leading to further breakdown in the extra-cellular matrix and the aneurysmal vessel. If this occurs, the excluded aneurysmal vessel may rupture leading to patient mortality. Therefore, certain embodiments of the present disclosure treat the aneurysmal sac 18 further in addition to, or alternative to, treating the proximal and/or distal neck regions.

In certain embodiments, the carrier is placed in a pouch that is attached to or wrapped around the outer—i.e., blood vessel wall side—of a stent graft passing through an aneurysmal blood vessel. The stent graft isolates the aneurysmal region of the blood vessel from blood flow and provides a structure on which to attach the delivery device so that the agent may be delivered directly to the aneurysmal blood vessel site. The delivery device is positioned on or wrapped around a stent, graft, stent graft, or other intervention device (all referred to herein as intravascular treatment devices) spanning the aneurysmal site through the interior of a blood vessel to release therapeutic agents into the space between the intervention device and the wall of the aneurysmal blood vessel. Devices of this type are disclosed, for example, in U.S. Patent Publication No. 2006/0004441, herein incorporated by reference.

### Therapeutic Agent Carrier

Two or more therapeutic agents are localized to (adjacent or within) the aneurysmal site. Preferably, this occurs by the placement of an intravascular treatment device that is comprised of, or within which is provided, the therapeutic agents. The therapeutic agents can be delivered by the intravascular treatment devices described herein in any of a variety of ways, several of which are described above. The therapeutic agents can be incorporated directly into an intravascular treatment device (e.g., incorporated into a polymer for forming a graft) or into a carrier associated with an intravascular treatment device (e.g., as a coating or in a pouch), or both.

The therapeutic agents can be mixed with, incorporated within, encased or enclosed within, a therapeutic agent carrier that can be made of one or more synthetic organic polymers, natural organic polymers, inorganics, or combinations (e.g., copolymers, mixtures, blends, layers, complexes, etc.) of these. The polymers may be biodegradable or non-biodegradable. The therapeutic agent/carrier formulation can be in the form of a film, sheet, threads, fibers (e.g., such as those used in making a graft material), coating (e.g., such as could be applied to a graft material), slab, gel, paste, capsule, microparticles or nanoparticles (e.g., such as could be included within a pouch), a pouch (e.g., in which the therapeutic agents can be placed), or combinations of these. Typically, the therapeutic agents are delivered by the intravascular treatment device over time to the local tissue. The carrier can be in a time-release formulation.

Protection of the therapeutic agents can also occur through the use of an inert molecule (e.g., in a cap- or over-coating over the therapeutic agents) that prevents access to the therapeutic agents. For example, a coating of the therapeutic agents can be over-coated readily with an enzyme, which causes either release of the therapeutic agents or activates the therapeutic agents. Alternating layers of the therapeutic coating with a protective coating may enhance the time-release properties of the coating overall. Thus, in certain embodiments, the treatment device can include least two therapeutic coatings, wherein each therapeutic coating is separated by a second coating.

The therapeutic agent/carrier formulation is preferably adapted to exhibit a combination of physical characteristics such as biocompatibility, and, in some embodiments, biodegradability and bio-absorbability, while providing a delivery vehicle for release of the therapeutic agents that aid in the treatment of aneurysmal tissue. For example, the formulation is preferably biocompatible such that it results in no induction of inflammation or irritation when implanted, degraded or absorbed.

Biodegradable materials include synthetic polymers such as polyesters, polyanhydrides, poly(ortho)esters, poly(butyric acid), tyrosine-based polycarbonates, poly(ester amide)s such as based on 1,4-butanediol, adipic acid, and 1,6-aminohexanoic acid, poly(ester urethane)s, poly(ester anhydride)s, poly(ester carbonate)s such as tyrosine-poly(alkylene oxide)-derived poly(ether carbonate)ₛ, polyphosphazenes, polyarylates such as tyrosine-derived polyarylates, poly(ether ester)s such as, poly(epsilon-caprolactone)-block-poly(ethylene glycol)) block copolymers, and poly(ethylene oxide)-block-poly(hydroxy butyrate) block copolymers.

Biodegradable polyesters, include, for example, poly(glycolic acid) (PGA), poly(lactic acid) (PLA), poly(glycolic-co-lactic acid) (PGLA), poly(1,4dioxanone), poly(caprolactone) (PCL), poly(3-hydroxybutyrate) (PHB), poly(3-hydroxyvalerate) (PHV), poly(hydroxy butyrate-co-hydroxy valerate), poly(lactide-co-caprolactone) (PLCL), poly(valerolactone) (PVL), poly(tartronic acid), poly(beta-malonic acid), poly(propylene fumarate) (PPF) (preferably photo cross-linkable), poly(ethylene glycol)/poly(lactic acid) (PELA) block copolymer, poly(L-lactic acid-epsilon-caprolactone) copolymer, poly(trimethylene carbonate), poly(butylene succinate), and poly(butylene adipate).

Biodegradable polyanhydrides include, for example, poly[1,6-bis(carboxyphenoxy)hexane], poly(fumaric-co-sebacic)acid or P(FA:SA), and such polyanhydrides used in the form of copolymers with polyimides or poly(anhydrides-co-imides) such as poly-[trimellitylimidoglycine-co-bis(carboxyphenoxy)hexane], poly[pyromellitylimidoalanine-co-1,6-bis(carboph-enoxy)-hexane], poly[sebacic acid-co-1,6-bis(p-carboxyphenoxy)hexane] or P(SA:CPH), poly[sebacic acids co-1,3-bis(p-carboxyphenoxy)propane] or P(SA:CPP), and poly(adipic anhydride).

Biodegradable materials include natural polymers and polymers derived therefrom, such as albumin, alginate, casein, chitin, chitosan, collagen, dextran, elastin, proteoglycans, gelatin and other hydrophilic proteins, glutin, zein and other prolamines and hydrophobic proteins, starch and other polysaccharides including cellulose and derivatives thereof (such as methyl cellulose, ethyl cellulose, hydroxypropyl cellulose, hydroxy-propyl methyl cellulose, hydroxybutyl methyl cellulose, carboxymethyl cellulose, cellulose acetate, cellulose propionate, cellulose acetate butyrate, cellulose acetate phthalate, cellulose acetate succinate, hydroxypropylmethylcellulose phthalate, cellulose triacetate, cellulose sulphate), poly-1-lysine, polyethylenimine, poly(allyl amine), polyhyaluronic acids, alginic acid, chitin, chitosan, chondroitin, dextrin or dextran), and proteins (such as albumin, casein, collagen, gelatin, fibrin, fibrinogen, hemoglobin).

Non-degradable (i.e., biostable) polymers include polyolefins such as polyethylene, polypropylene, polyurethanes, fluorinated polyolefins, such as polytetrafluorethylene, chlorinated polyolefins such as poly(vinyl chloride), polyamides, acrylate polymers such as poly(methyl methacrylate), acrylamides such as poly(N-isopropylacrylamide), vinyl polymers such as poly(N-vinylpyrrolidone), poly(vinyl alcohol), poly(vinyl acetate), and poly(ethylene-co-vinylacetate), polyacetals, polycarbonates, polyethers such as based on poly(oxyethylene) and poly(oxypropylene) units, aromatic polyesters such as poly(ethylene terephthalate) and poly(propylene terephthalate), poly(ether ether ketone)s, polysulfones, silicone rubbers, epoxies, and poly(ester imide)s.

Representative examples of inorganics include hydroxyapatite, tricalcium phosphate, silicates, montmorillonite, and mica.

Preferred biodegradable polymers include polymers of lactide, caprolactone, glycolide, trimethylene carbonate, p-dioxanone, gamma-butyrolactone, or combinations thereof in the form of random or block copolymers. Preferred non-biodegradable polymers include polyesters, polyamides, polyurethanes, polyethers, vinyl polymers, and combinations thereof.

Particularly preferred polymers include the following: a polymer with phosphoryl choline functionality to encourage ionic interactions, including but not limited to methacrylate copolymer with MPC comonomer (Formula I); a polymer with multiple hydroxyl groups encouraging hydrogen bonding interaction with the therapeutic agents, including but not limited to that shown in Formula II; a polymer with acidic or basic groups encouraging acid-base interaction with the therapeutic agents, including but not limited to those shown in Formulas III and IV.

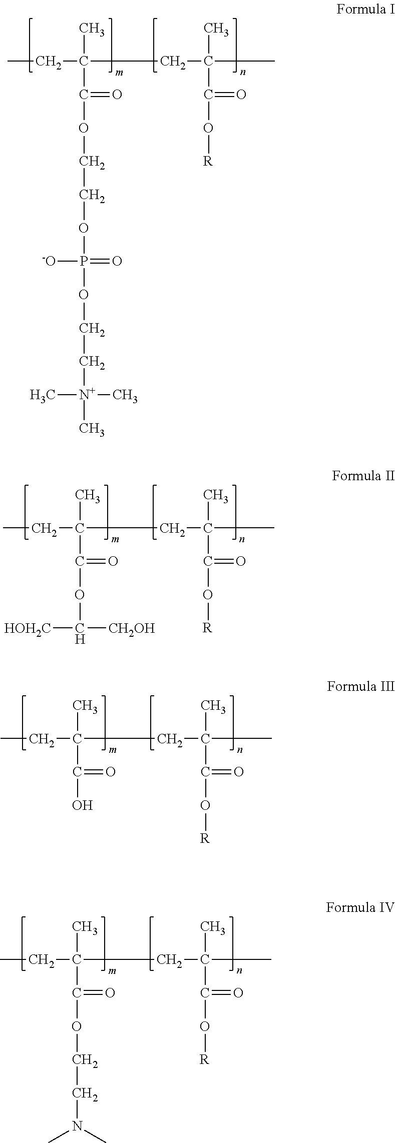

In the above formulas (I through IV), the R groups are independently C1 to C20 straight chain alkyl, C3 to C8 cycloalkyl, C2 to C20 alkenyl, C2 to C20 alkynyl, C2 to C14 heteroatom substituted alkyl, C2 to C14 heteroatom substituted cycloalkyl, C4 to C10 substituted aryl, or C4 to 010 substituted heteroatom substituted heteroaryl. In certain embodiments, m and n are individually integers from 1 to 20,000. In certain embodiments, m is an integer ranging from 10 to 20,000; from 50 to 15,000; from 100 to 10,000; from 200 to 5,000; from 500 to 4,000; from 700 to 3,000; or from 1000 to 2000. In certain embodiments, m is an integer ranging from 10 to 20,000; from 50 to 15,000; from 100 to 10,000; from 200 to 5,000; from 500 to 4,000; from 700 to 3,000; or from 1000 to 2000.

Particularly preferred polymers are shown below in Formulas V and VI:

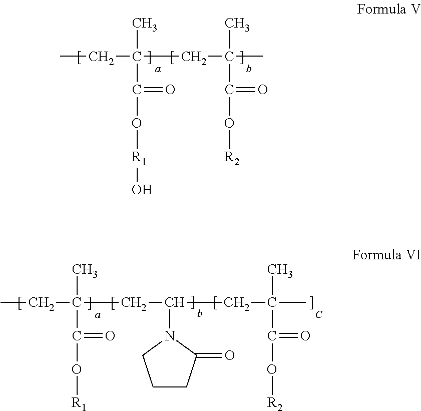

In the above formulas (V and VI), the R1 and R2 groups are independently C1 to 020 straight chain alkyl, C3 to C8 cycloalkyl, C2 to C20 alkenyl, C2 to C20 alkynyl, C2 to C14 heteroatom substituted alkyl, C2 to C14 heteroatom substituted cycloalkyl, C4 to C10 substituted aryl, or C4 to C10 substituted heteroatom substituted heteroaryl. In certain embodiments, a is an integer ranging from 10 to 20,000; from 50 to 15,000; from 100 to 10,000; from 200 to 5,000; from 500 to 4,000; from 700 to 3,000; or from 1000 to 2000. In certain embodiments, b is an integer ranging from 10 to 20,000; from 50 to 15,000; from 100 to 10,000; from 200 to 5,000; from 500 to 4,000; from 700 to 3,000; or from 1000 to 2000. In certain embodiments, c is an integer ranging from 10 to 20,000; from 50 to 15,000; from 100 to 10,000; from 200 to 5,000; from 500 to 4,000; from 700 to 3,000; or from 1000 to 2000.

The polymer(s) used may be obtained from various chemical companies known to those with skill in the art. However, because of the presence of unreacted monomers, low molecular weight oligomers, catalysts, and other impurities, it may be desirable (and, depending upon the materials used, may be necessary) to increase the purity of the polymer used. The purification process yields polymers of better-known, purer composition, and therefore increases both the predictability and performance of the mechanical characteristics of the coatings. The purification process will depend on the polymer or polymers chosen. Generally, in the purification process, the polymer is dissolved in a suitable solvent. Suitable solvents include (but are not limited to) methylene chloride, ethyl acetate; chloroform, and tetrahydrofuran. The polymer solution usually is then mixed with a second material that is miscible with the solvent, but in which the polymer is not soluble, so that the polymer (but not appreciable quantities of impurities or unreacted monomer) precipitates out of solution. For example, a methylene chloride solution of the polymer may be mixed with heptane, causing the polymer to fall out of solution. The solvent mixture then is removed from the copolymer precipitate using conventional techniques.

In certain embodiments described herein, the therapeutic agent/carrier formulation comprises a material to ensure the controlled release of the therapeutic agent. The materials to be used for such a formulation—as well as the delivery vehicle itself, in some embodiments—are preferably comprised of a biocompatible polymer, in which the therapeutic agent is present. A dispersion of a therapeutic agent in a carrier, for example, allows the therapeutic reaction to be substantially localized so that overall dosages to the individual can be reduced, and undesirable side effects caused by the action of the agent in other parts of the body are minimized. The carrier can be in the form of a polymer coating, for example.

The therapeutic agents may be linked by occlusion in the matrices of the polymer coating, bound by covalent linkages to the coating or to a biodegradable stent, or encapsulated in microcapsules that are associated with the stent and are themselves biodegradable.

In certain embodiments, the therapeutic agent/carrier formulation is formulated to deliver the therapeutic agents over a period of several hours, days, or, months. For example, “quick release” or “burst” coatings are provided that release greater than 10%, 20%, or 25% (w/v) of the therapeutic agents over a period of 7 to 10 days. Within other embodiments, “slow release” therapeutic agents are provided that release less than 10% (w/v) of a therapeutic agent over a period of 7 to 10 days. Further, the therapeutic agents of the present disclosure preferably should be stable for several months and capable of being produced and maintained under sterile conditions.

In certain embodiments, therapeutic coatings may be fashioned in any thickness ranging from about 50 nm to about 3 mm, depending upon the particular use. Alternatively, such compositions may also be readily applied as a “spray”, which solidifies into a film or coating. Such sprays may be prepared from microspheres of a wide array of sizes, including for example, from 0.1 micron to 3 microns, from 10 microns to 30 microns, and from 30 microns to 100 microns.

The therapeutic agents of the present disclosure also may be prepared in a variety of “paste” or gel forms. For example, within one embodiment of the disclosure, therapeutic coatings are provided which are liquid at one temperature (e.g., temperature greater than 37° C., such as 40° C., 45° C., 50° C., 55° C. or 60° C.), and solid or semi-solid at another temperature (e.g., ambient body temperature, or any temperature lower than 37° C.). Such “thermopastes” readily may be made utilizing a variety of techniques. Other pastes may be applied as a liquid, which solidify in vivo due to dissolution of a water-soluble component of the paste.

In other embodiments, the therapeutic compositions of the present disclosure may be formed as a film. Preferably, such films are generally less than 5, 4, 3, 2, or 1 mm thick, more preferably less than 0.75 mm, 0.5 mm, 0.25 mm, or, 0.10 mm thick. Films can also be generated of thicknesses less than 50 microns, 25 microns or 10 microns. Such films are preferably flexible with a good tensile strength (e.g., greater than 50, preferably greater than 100, and more preferably greater than 150 or 200 N/cm²), have good adhesive properties (i.e., adhere to moist or wet surfaces), and have controlled permeability.

### EXAMPLES

Objects and advantages of this disclosure are further illustrated by the following examples, but the particular materials and amounts thereof recited in these examples, as well as other conditions and details, should not be construed to unduly limit this disclosure.

The ApoE^{-/-}+Ang II AAA model is well established and supported by current scientific literature. Mice genetically predisposed to hypercholesterolemia develop aneurysms when treated with angiotensin II. Aneurysms generally develop within the first week after pump implantation and share many important pathologic characteristics with human AAA disease.

### Angiotensin II Infusion

ApoE−/− mice used in this study were divided into different pretreatment groups: 1) Fluvastatin-MF; 2) Doxycycline-MD; 3) Irbesartan-MI; 4) Telmisartan-MT; 5) Control (water) and fed medicated chow or medicated drinking water 1 week prior to pump implantation. AngII (1000 ng·kg⁻¹ of body weight·min⁻¹) was infused subcutaneously via Alzet mini-osmotic pumps for 28 days. All mice were maintained on the medicated chow (MI & MT) or medicated drinking water (MF & MD) treatments which were delivered daily for a total of 28 days following pump implantation.

In order to determine the effect of the various treatments on the progressive enlargement of the experimental AAAs the mice were followed with high frequency trans-abdominal ultrasound. Maximum aortic diameter was measured and recorded prior to pump implantation and then at 3, 7, 14, 21, and 28 days. All mice were sacrificed 28 days after pump implantation. Aortae were harvested and subjected to either histological analysis (Elastic Masson trichome, MAC2, CD31 and TUNEL staining) and analysis of mural gene expression. Plasma drug concentrations of experimental compounds were quantified on the day of sacrifice using high performance liquid chromatography (HPLC).

Ribonucleic acid (RNA) Extraction, Complimentary Deoxyribonucleic Acid Synthesis

Total RNA was extracted from homogenized aortas using the Qiazol lysis reagent and RNeasy Lipid Tissue Mini kit. DNAse treatment was included in the procedure. RNA concentration was determined utilizing the Quant iT RNA assay kit. Complimentary deoxyribonucleic acid (cDNA) was synthesized from 1 μg RNA using High reverse cDNA transcription kit. Briefly the reaction tubes containing RNA, reverse transcriptase, buffer, RNAse inhibitor, and nuclease free water were incubated at 25° C. for 10 minutes to allow annealing. Reverse transcription was performed at 37° C. for 120 min, followed by a 5 sec incubation at 85° C. to inactivate the reverse transcriptase enzyme. The cDNA samples were cooled at 4° C. and stored at −80° C. until further use.

Quantitative-Real Time Polymerase Chain reaction (RT-PCR)

Real time PCR was performed using a 384-well Taqman low density array (TLDA) card. Each sample specific PCR mix contained 50 ng of total RNA converted to cDNA. The PCR reaction for Taqman assays contained 50 μl Master Mix (Taqman Universal PCR Master Mix,) and 50 ul of cDNA and RNAase free water. Assays were performed to include appropriate controls (no template control). The RT-PCR protocol included an initial step of 50° C. (2 minutes) to activate the DNA polymerase, denaturation by a hot start at 95° C. for 10 min, followed by 40 cycles of a 2 step program (denaturation at 95° C. for 15 secs for primer annealing/extension at 60° C. for 1 min). Fluorescence data was collected at 60° C. Fluorescence was quantified with ABI PRISM 7900HT Sequence Detection System. To verify amplification of a specific target cDNA, data generated was analyzed using SDS 2.2.3 software (Sequence Detection System Software, Applied Biosystems). For all amplification plots, the baseline data were set with the automatic C_{T} function available with SDS 2.2.3 calculating the optimal baseline range and threshold value by using the AutoC_{T} algorithm. According to the manufacturer's instruction, a C_{T} value of ≧39 corresponds to nonspecific amplification marking the limits of detection. For endogenous control GAPDH was normalized to B2M. For relative quantification (RQ), pooled ApoE−/− control was used as the calibrator (with expression equal to 1).

### Discussion:

The pathological features of AAA are characterized by chronic inflammation, destruction of the elastic media, revascularization, and depletion of the vascular smooth muscle cells. A number of molecular mediators and extracellular matrix-degrading proteinases contribute to the pathological process of aortic wall degradation, and the histologically changes in the aneurysm wall are believed to result from the complex interactions among these factors.

Chronic inflammation of the aortic wall plays a pivotal role in the pathogenesis of AAA. Studies of human AAA have shown extensive inflammatory infiltrates containing macrophages and lymphocytes in both the media and adventitia and increasing aneurysm diameter was associated with a higher density of inflammatory cells in the adventitia. Activated macrophages are the culprit responsible for secreting various proteases leading to the disruption of the orderly lamellar structure of the aortic media. Angiotensin (Ang) II is considered to be one of the factors inducing aortic inflammation. Ang II is the main effector peptide in the renin—angiotensin system (RAS) and exerts pro-inflammatory actions through an increase in the expression of several mediators including leukocyte adhesion molecules and chemokines. Sustained infusion of Ang II leads to aneurysmal lesions in the atherosclerosis-prone ApoE^{-/-} mouse. There is increasing evidence on the importance of tissue RAS in the vasculature. Therefore, Ang II has emerged as a central factor in the initiation and progression of AAA and potential target for treating AAA.

Proteolysis of extracellular matrix proteins plays an important role in aneurysm development and involves a complex remodeling process with an imbalance between the synthesis and degradation of connective tissue proteins. Various extracellular proteinases participate in the process of the destruction of the human aortic wall in particular, MMPs are considered to be the predominant proteinases. Several MMPs have been focused on in AAA, including four that degrade elastic fibers (MMP-2, MMP-7, MMP-9, and MMP-12), several that degrade interstitial collagen (MMP-1, MMP-2, MMP-8, MMP-13, and MMP-14), and others that degrade denatured collagen (MMP-2 and MMP-9). Cathepsins are another class of proteases reported to also contribute to the initiation and progression of AAA. Cathepsins are members of cysteine proteases and are regulated by the inhibitor cystatin C. It has been shown that the activities of cathepsin B, H, L, and S were significantly higher, and the level of cystatin C was lower in the aneurysm wall than in the aortic wall of occlusive aortic disease. Therefore, extracellular matrix degrading proteases has emerged as a central factor in the initiation and progression of AAA and potential target for treating AAA.

Oxidative stress has been associated with the formation of AAA. Several stimuli enhance reactive oxygen species (ROS) and reactive nitrogen species (RNS) production, leading to cell and tissue damage in many physiological conditions. In human studies, ROS and RNS were increased in the aneurysm wall compared with the normal aorta and adjacent non-aneurysmal aortic wall. Infiltrated inflammatory cells are the main source of ROS production such as O₂⁻ and H₂O₂ through the upregulated activity of NADPH oxidase. In addition, pro-inflammatory cytokines, mechanical stretch, growth factors, and lipid mediators might upregulate NADPH oxidase in resident vascular cells, resulting in an increase in the production of ROS and lipid peroxidation products. ³Overexpressed ROS and NO increased the expression of MMPs through the activation of nuclear factor-kappaB (NFκB) and induced apoptosis of VSMC in the aneurysm wall.

Transcriptional profiling shows genes significantly (p≦0.05) regulated in the ApoE^{-/-} angiotensin II (Ang II) model relevant to aneurysm formation (Table 1). These categories of these genes fall within the following classes (i) inflammatory cytokines and their receptors (ii) protease for ECM degradation, (iii) oxidative stress, (iv) cell adhesion molecule, (v) transcription factors, and (vi) T cell activation and signaling.

Drug inhibition (Table 2) shows the efficacy of telmisartan, irbesartan and fluvastatin in down-regulating the expression of genes involved in the pathophysiology of AAA in this experimental model. Down regulation of these genes resulted in inhibition and reduction in aneurysm formation in the apolipoprotein E-deficient (ApoE^{-/-})+angiotensin II (Ang II) AAA model. The molecular pathways shown to be affected by these drugs are implicated in inflammation, matrix metalloproeinase, cathepsins, reactive oxygen species (ROS) production, cell adhesions molecule.

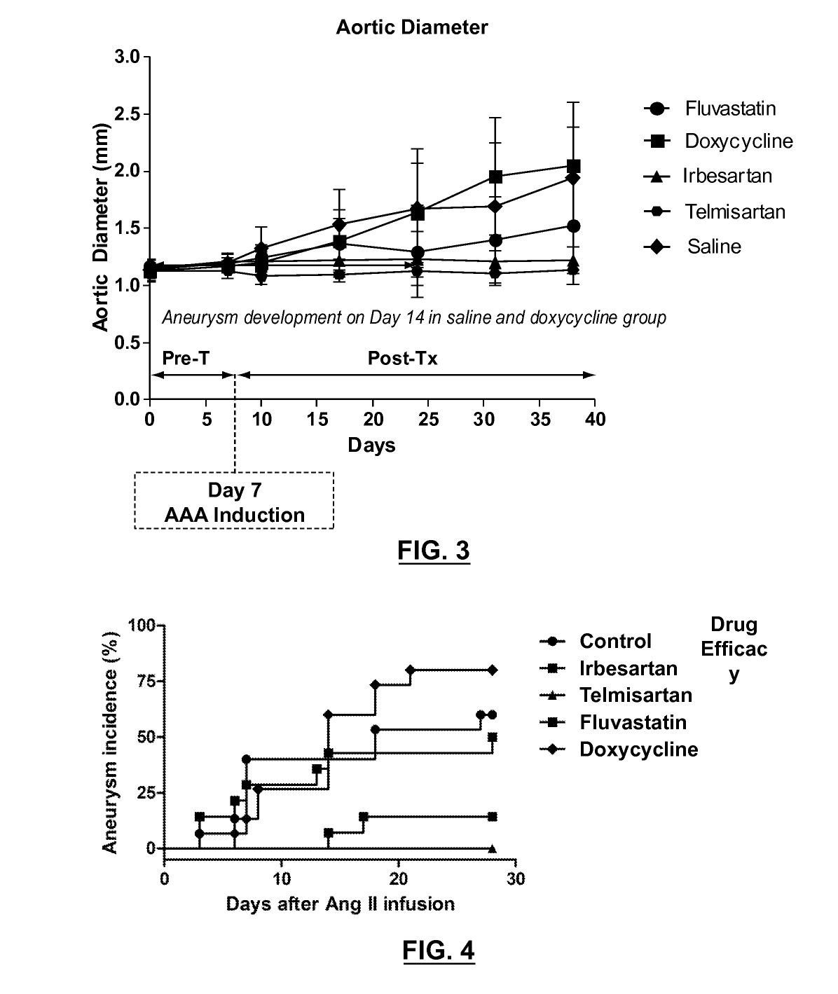

Aortic diameter measurements (FIG. 3) by ultrasonography demonstrate that pretreatment of mice with telmisartan and irbesartan prior to ANG II infusion was effective in inhibiting/preventing aneurysm development. Fluvastatin showed a relative good inhibition of AAA when compared to saline control and doxycycline. Doxycycline historically used as an inhibitor of AAA; did not inhibit AAA growth in this model.

FIG. 4 shows the relationship between aneurysm incidence and passage of time. Aneurysm was defined as either the presence of dissection or more than 50% increase in diameter. Results show that telmisartan, irbesartan, and fluvastatin were effective in inhibiting aneurysm development compared to doxycycline and saline control group.

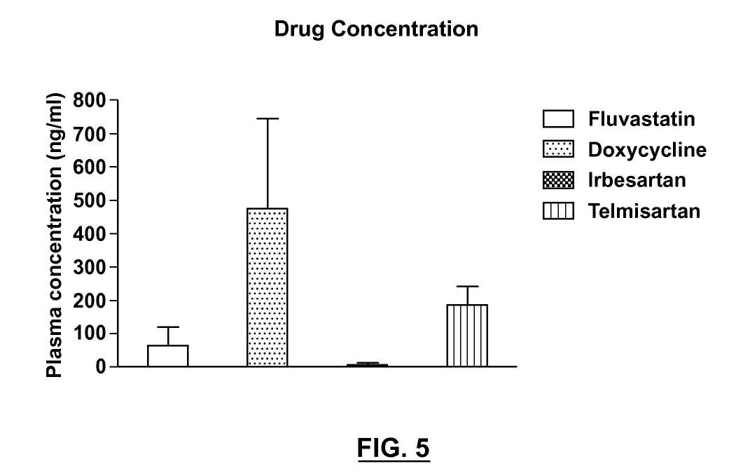

The plasma drug content of the various compounds in each treatment group, was assessed by high performance liquid chromatography (HPLC). FIG. 5 shows all treatment group had detectable amount of drug in their blood stream. The lower plasma concentration of Irbesartan compared to telmisartan may be attributed to differences in the protein binding or receptor binding, or half-life differences between irbesartan and telmisartan.

TABLE 1
Summary of Disease Relevant Genes Common
to Both Infrarenal and Suprarenal Aorta

| Function | Symbol | Gene Name |
|---|---|---|
| Inflammatory | ↑ CCL2 | Chemokine (C-C motif) ligand 2 |
| Cytokines | ↑ CCR2 | Chemokine (C-C motif) receptor 2 |
| and Receptors | ↑ CCR5 | Chemokine (C-C motif) receptor 5 |
| | ↑ CXCR4 | Chemokine (C-X-C motif) |
| | | receptor 4 |
| | ↑ SPP1 | Secreted phosphoprotein 1 |
| | ↑ TNFALPHA | Tumor necrosis factor-ALPHA |
| Protease and ECM | ↑ MMP 8 | Matrix metallopeptidase 8 |
| Degradation | ↑ MMP 12 | Matrix metallopeptidase 12 |
| | ↑ CTSB | Cathepsin B |
| | ↑ CTSS | Cathepsin S |
| Protection Against | ↑ HMOX1 | Heme oxygenase (decycling) 1 |
| Inflammation and | ↑ CYBB | Cytochrome b-245 |
| Oxidative Stress | (NOX-2) | |
| Cell Adhesion | ↑ ITGAL | Integrin alpha L |
| Molecule | ↑ ITGB2 | Integrin beta 2 |
| Select regulatory | ↑ RAC2 | RAS-related C3 botulinum |
| molecule, G-protein, | | substrate 2 |
| Small GTPase | | |
| Transcription Factor | ↑ RUNX3 | Runt related transcription factor 3 |
| T Cell Activation | ↑ VAV1 | vav 1 oncogene |
| Signaling Molecule | ↓ CSF1 | Colony stimulating factor 1 |
| Kinase | ↓ MAPK8 | Mitogen-activated protein kinase 8 |

Table 1 show genes significantly (p≦0.05) regulated in the ApoE^{-/-} angiotensin II (Ang II) model relevant to aneurysm formation. These genes fall within the following categories (i) inflammatory cytokines and their receptors (ii) protease for ECM degradation, (iii) oxidative stress, (iv) cell adhesion molecule, (v) transcription factors, and (vi) T cell activation and signaling.

TABLE 2
Drug Inhibition Effect on Differentially Expressed Genes

| Gene | ApoE⁻/⁻ + | Doxycy- | Fluva- | | |
|---|---|---|---|---|---|
| Symbol | Ang II | cline | statin | Irbesartan | Telmisartan |
| Osteopontin | ++++ | ++++ | ++++ | ++ | − |
| MMP-8 | ++++ | ++++ | ++++ | ++ | − |
| MMP-12 | ++++ | ++++ | +++ | +++ | − |
| CXCR4 | ++++ | ++++ | +++ | − | +++ |
| Cathepsin S | ++++ | +++ | +++ | − | − |
| HMOX 1 | +++ | ++++ | +++ | − | − |
| Tgfβ1 | +++ | +++ | +++ | − | − |
| CCR2 | +++ | +++ | +++ | − | − |
| CCR5 | +++ | +++ | +++ | − | − |
| Runx3 | +++ | +++ | ++ | − | − |
| TNF-α | +++ | ++ | ++ | − | − |
| CCL2 | +++ | + | ++ | − | − |
| Cybb | ++ | +++ | ++ | − | − |
| ITGAL | ++ | +++ | ++ | − | |
| Rac2 | ++ | +++ | + | − | − |
| ITGB2 | ++ | ++ | ++ | − | − |
| Vav1 | ++ | ++ | ++ | − | − |
| Cathepsin B | ++ | ++ | + | − | − |
| IL-7 | ++ | − | − | − | + |
| MAPK8 | − | − | − | − | ++ |
| LOX | − | − | − | − | − |
| CSF1 | − | − | − | − | − |
| NFKb1 | − | − | − | − | − |
| Fibronec- | − | − | − | − | − |
| tin -1 | | | | | |
| TIMP2 | − | − | − | − | − |
| Total Score | 55 | 53 | 45 | 7 | 6 |

Heat Map Key: Fold change over ApoE⁻/⁻ control
>10.1 = ++++
3.1 to 10 = +++
1.51 to 3 = ++
1.1 to 1.5 = +
0 to 1 = −

Table 2 shows the efficacy of two ARB's and a statin in down-regulating the expression of genes involved in the pathophysiology of AAA in this experimental model resulting in inhibiting and reducing aneurysm formation in the apolipoprotein E-deficient (ApoE^{/-}) angiotensin II (Ang II) AAA model. Molecular pathways shown to be affected by these compounds are implicated in inflammation, MMP's, cathepsins, ROS production, cell adhesions molecule.

The complete disclosures of all patents, patent applications, publications, and nucleic acid and protein database entries, including for example GenBank accession numbers and EMBL accession numbers, that are cited herein are hereby incorporated by reference as if individually incorporated. Various modifications and alterations of this disclosure will become apparent to those skilled in the art without departing from the scope and spirit of this disclosure, and it should be understood that this disclosure is not to be unduly limited to the illustrative embodiments set forth herein.

1. A method of treating a vascular aneurysm in a subject, the method comprising:
providing an intravascular treatment device comprising two or more therapeutic agents, wherein the two or more therapeutic agents comprise:
at least one HMG-CoA reductase inhibitor; and
at least one of a second therapeutic agent selected from the group consisting of an ACE inhibitor, an Angiotensin II Receptor Blocker, a calcium channel blocker, a renin inhibitor, a prostanoid receptor antagonist, a cholesterol absorption inhibitor, and combinations thereof; and positioning the intravascular treatment device in the interior of an aneurysmal site in a blood vessel, wherein the intravascular treatment device supports the aneurysmal site upon deployment.

2. The method of claim 1 wherein the vascular aneurysm is an abdominal aortic aneurysm.

3. The method of claim 1 wherein the two or more therapeutic agents are associated the intravascular treatment device such that when the device is positioned in the interior of the aneurysmal site, the two or more therapeutic agents are in the proximal neck of the aneurysm.

4. The method of claim 1, wherein the intravascular treatment device comprises a polymeric coating comprising the two or more therapeutic agents.

5. The method of claim 1, wherein the intravascular treatment device comprises a structural polymeric component comprising the two or more therapeutic agents.

6. The method of claim 1, wherein the intravascular treatment device comprises a mixture of the two or more therapeutic agents.

7. The method of claim 1, wherein the intravascular treatment device comprises a stent graft.

8. The method of any one of claim 1 wherein the HMG-CoA reductase inhibitor is a statin.

9. The method of claim 8 wherein the statin is selected from the group consisting of a lovastatin, cerivastatin, pitavastatin, pravastatin, fluvastatin, rosuvastatin, simivastatin, atorvastatin, their physiologically active metabolites, and combinations thereof.

10. The method of claim 1, wherein the ACE inhibitor is selected from the group consisting of trandolapril, lisinopril, enalapril, ramipril, fosinopril, cilazapril, imidapril, captopril, quinapril, perindopril, benazepril, moexipril, their physiologically active metabolites, and combinations thereof.

11. The method of claim 1, wherein the Angiotensin II Receptor Blocker is selected from the group consisting of irbestartan, candesartan, losartan, valsartan, telmisartan, eprosartan, olmesartan, their physiologically active metabolites, and combinations thereof.

12. The method of claim 1, wherein the calcium channel blocker is a dihydropyridine calcium channel blocker.

13. The method of claim 12 wherein the dihydropyridine calcium channel blocker is selected from the group consisting of amlodipine, aranidipine, azelnidipine, barnidipine, benidipine, cilnidipine, clevidipine, isradipine, efonidipine, felodipine, lacidipine, lercanidipine, manidipine, nicardipine, nifedipine, nilvadipine, nimodipine, nisoldipine, nitrendipine, pranidipine, their physiologically active metabolites, and combinations thereof.

14. The method of claim 1, wherein the renin inhibitor is selected from the group consisting of aliskiren, remikiren, enalkiren, MK8141, their physiologically active metabolites, and combinations thereof.

15. The method of claim 1, wherein the prostanoid receptor antagonist is a DP1 receptor antagonist.

16. The method of claim 15 wherein the DP1 receptor antagonist is laropiprant, an azaindole, their physiologically active metabolites, and combinations thereof.

17. The method of claim 1, wherein the a cholesterol absorption inhibitor is selected from the group consisting of ezetimibe, niacin, and Niemann-Pick Cl-Like 1 (NPC1L1) inhibitors, their physiologically active metabolites, and combinations thereof.

18. The method of claim 1, wherein the intravascular treatment device further comprises a carrier for the therapeutic agents.

19. The method of claim 18 wherein the carrier comprises an organic polymeric material.

20. The method of claim 19 wherein the organic polymeric material is non-biodegradable.

21. An intravascular treatment device locatable interior of an aneurysmal site in a blood vessel; wherein the device supports the aneurysmal site upon deployment, contracts when the aneurysmal site contracts, and comprises two or more therapeutic agents, wherein the two or more therapeutic agents comprise:
at least one HMG-CoA reductase inhibitor; and
at least one of a second therapeutic agent selected from the group consisting of an ACE inhibitor, an Angiotensin II Receptor Blocker, a calcium channel blocker, a renin inhibitor, a prostanoid receptor antagonist, a cholesterol absorption inhibitor, and combinations thereof.

22. The device of claim 21 wherein the intravascular treatment device comprises a stent graft.

23. The device of claim 21 wherein the two or more therapeutic agents are associated with the intravascular treatment device such that when the device is positioned in the interior of the aneurysmal site, the two or more therapeutic agents are in the proximal neck of the aneurysm.

24. The device of claim 21, wherein the intravascular treatment device comprises a polymeric coating comprising the two or more therapeutic agents.

25. The device of claim 21, wherein the intravascular treatment device comprises a structural polymeric component comprising the two or more therapeutic agents.

26. The device of claim 21, wherein the intravascular treatment device comprises a mixture of the two or more therapeutic agents.

27. The device of claim 21, wherein the intravascular treatment device further comprises a carrier for the therapeutic agents.

28. The device of claim 27 wherein the carrier comprises an organic polymeric material.

29. The device of claim 21, wherein the HMG-CoA reductase inhibitor is a statin.

30. The device of claim 21, wherein:
the ACE inhibitor is selected from the group consisting of trandolapril, lisinopril, enalapril, ramipril, fosinopril, cilazapril, imidapril, captopril, quinapril, perindopril, benazepril, moexipril, their physiologically active metabolites, and combinations thereof;
the Angiotensin II Receptor Blocker is selected from the group consisting of irbestartan, candesartan, losartan, valsartan, telmisartan, eprosartan, olmesartan, their physiologically active metabolites, and combinations thereof;
the calcium channel blocker is selected from the group consisting of amlodipine, aranidipine, azelnidipine, barnidipine, benidipine, cilnidipine, clevidipine, isradipine, efonidipine, felodipine, lacidipine, lercanidipine, manidipine, nicardipine, nifedipine, nilvadipine, nimodipine, nisoldipine, nitrendipine, pranidipine, their physiologically active metabolites, and combinations thereof;
the renin inhibitor is selected from the group consisting of aliskiren, remikiren, enalkiren, MK8141, their physiologically active metabolites, and combinations thereof;
the prostanoid receptor antagonist is laropiprant, an azaindole, their physiologically active metabolites, and combinations thereof and
the a cholesterol absorption inhibitor is selected from the group consisting of ezetimibe, niacin, and Niemann-Pick Cl-Like 1 (NPC1L1) inhibitors, their physiologically active metabolites, and combinations thereof.
